(19) United States
(12) Patent Application Publication
(10) Pub. No.: US 2011/0245630 A1
(43) Pub. Date: Oct. 6, 2011

(54) Integrated Patient Data Management for Physiological Monitor Devices

(76) Inventors: Shawn C. St. Pierre, Syracuse, NY (US); Michael D. Garrant, Marcellus, NY (US); Kristin Ann Alisanski, Syracuse, NY (US)

(21) Appl. No.: 12/751,602

(22) Filed: Mar. 31, 2010

(51) Int. Cl. A61B 5/00; G06Q 50/00; G06Q 10/00

(52) U.S. Cl. 600/301; 705/3; 235/380

(57) ABSTRACT

A physiological monitor device is programmed to: store a device ID for the device, the device ID being a number that uniquely identifies the device, the device being configured with the device ID by a user; store a location ID for the device, the location ID identifying a location in a medical facility, the device being configured with the location ID by a user; send the device ID and the location ID to a server computer; after the device ID and the location ID are sent to the server computer, receive a list of patients for the location specified by the location ID; and display the list of patients on the device.

### BACKGROUND

Health care practitioners, such as nurses and physicians, use various types of health-care equipment to assist with the task of providing health care to a patient, also referred to herein as a health-care recipient. Some health-care equipment, referred to as single function equipment, is designed to perform a particular function, such as temperature measurement. Some health-care equipment, referred to as multi-function equipment, is designed to implement the performance of more than one function, such as temperature measurement and blood pressure measurement. Such multi-function equipment may impose excess bulk and/or weight upon a user if such multi-function equipment is used for only one function or a subset of the functions implemented by the multi-function equipment.

### SUMMARY

In one aspect, a physiological monitor device includes a central processing unit (CPU) that is configured to control operation of the device, a display screen, and a set of one or more computer readable data storage media storing software instructions that, when executed by the CPU, cause the device to: store a device ID for the device, the device ID being one or more characters that uniquely identifies the device, the device being configured with the device ID by a user; store a location ID for the device, the location ID identifying a location in a medical facility, the device being configured with the location ID by a user; send the device ID and the location ID to a server computer; after the device ID and the location ID are sent to the server computer, receive a list of patients for the location specified by the location ID; and display the list of patients on the device.

In yet another aspect, a method for reviewing and processing physiological measurement records includes: storing a device ID for a physiological monitor device, the device ID being a number that uniquely identifies the physiological monitor device, the physiological monitor device being configured with the device ID by a user; storing a location ID for the physiological monitor device, the location ID specifying a floor or a room number in a medical facility, the physiological monitor device being configured with the location ID by a user; sending the device ID and the location ID to a server computer; after the device ID and the location ID are sent to the server computer, receiving a list of patients at the location specified by the location ID; and displaying the list of patients on the physiological monitor device.

In another aspect, a computer-readable storage medium includes software instructions that, when executed, cause a physiological monitor device to: store a device ID for the device, the device ID being a number that uniquely identifies the device, the device being configured with the device ID by a user; store a location ID for the device, the location ID specifying a floor or a room number in a medical facility, the device being configured with the location ID by a user; send the device ID and the location ID to a server computer; after the device ID and the location ID are sent to the server computer, receiving a list of patients at the location specified by the location ID; and display the list of patients on the device; select a patient from the list of patients; after a patient is selected, display a home screen on the device, the home screen displaying current physiological measurement readings for the patient, the home screen including at least two identifiers for the patient, the at least two identifiers including two of a patient ID, the name of the patient and a location ID for the patient; select a review tab on the device; when the review tab is selected, display a list of physiological measurement records for the patients at the location specified by the location ID, each physiological measurement record in the list of physiological measurement records including a patient name, at least one patient name being abbreviated; select one or more of the physiological measurement records and sending the physiological measurement records to a server computer; and automatically delete the selected physiological measurement records from the list of physiological measurement records after the selected physiological measurement records are sent to the server computer.

In yet another aspect, a method for identifying a patient on a physiological monitor device includes: scanning a barcode on a wristband attached to the patient, the barcode being scanned using a barcode scanning device; sending the scanned barcode to a server computer; receiving, at the physiological monitor device, a patient ID and a name of the patient associated with the scanned barcode; and displaying, on the physiological monitor device, both the patient ID and the patient name.

### BRIEF DESCRIPTION OF THE DRAWINGS

The present disclosure can be better understood with reference to the claims and drawings described below. The drawings are not necessarily to scale, and the emphasis is instead generally being placed upon illustrating the principles of the invention. Within the drawings, like reference numbers are used to indicate like parts throughout the various views. Differences between like parts may cause those like parts to be each indicated by different reference numbers. Unlike parts are indicated by different reference numbers.

FIG. 1 is a block diagram illustrating an example system for collecting measurements of physiological parameters of patients.

FIG. 2A illustrates a view of an example physiological monitor device.

FIG. 2B illustrates an example user interface displayed on a user interface display of the physiological monitor device of FIG. 2A.

FIG. 3 illustrates an example patient list screen.

FIG. 4 illustrates an example patient summary screen.

FIG. 5 illustrates an example patient identifier advanced information screen.

FIG. 6 illustrates an example home screen.

FIG. 7 illustrates an example patient monitor screen.

FIG. 8 illustrates an example screen for manually entering patient information.

FIG. 9 illustrates an example data management display screen.

FIG. 10 illustrates an example advanced settings screen.

FIG. 11 illustrates an example device settings screen.

FIG. 12 illustrates an example clinician identifier advanced information screen.

FIG. 13 illustrates an example review screen.

FIG. 14 illustrates a data management screen for clinical data.

FIG. 15 is a flowchart illustrating example operations performed by the physiological monitor device.

FIG. 16 is a flowchart illustrating another example operation performed by the physiological monitor device.

FIG. 17 illustrates example physical components of the physiological monitor device.

FIG. 18 illustrates another example patient list screen.

FIG. 19 illustrates an example patient record screen.

### DETAILED DESCRIPTION

Embodiments of the present disclosure are directed to a physiological monitor device having a user interface configured to operate within and transition between each of a monitoring workflow and a non-monitoring workflow. The user interface includes features that protect the privacy of patients and that facilitate patient identification and patient data management.

In some examples described herein, the physiological monitor device is a portable device. In other examples, the physiological monitor device is a non-portable device, such as a computing device like a workstation. Many configurations are possible.

In the following detailed description, references are made to the accompanying drawings that form a part hereof, and in which are shown by way of illustrations specific embodiments or examples. These embodiments may be combined, other embodiments may be utilized, and structural changes may be made without departing from the spirit or scope of the present invention. The following detailed description is therefore not to be taken in a limiting sense and the scope of the present invention is defined by the appended claims and their equivalents.

Referring now to the drawings, in which like numerals refer to like elements through the several figures, aspects of the present invention and an exemplary computing operating environment will be described.

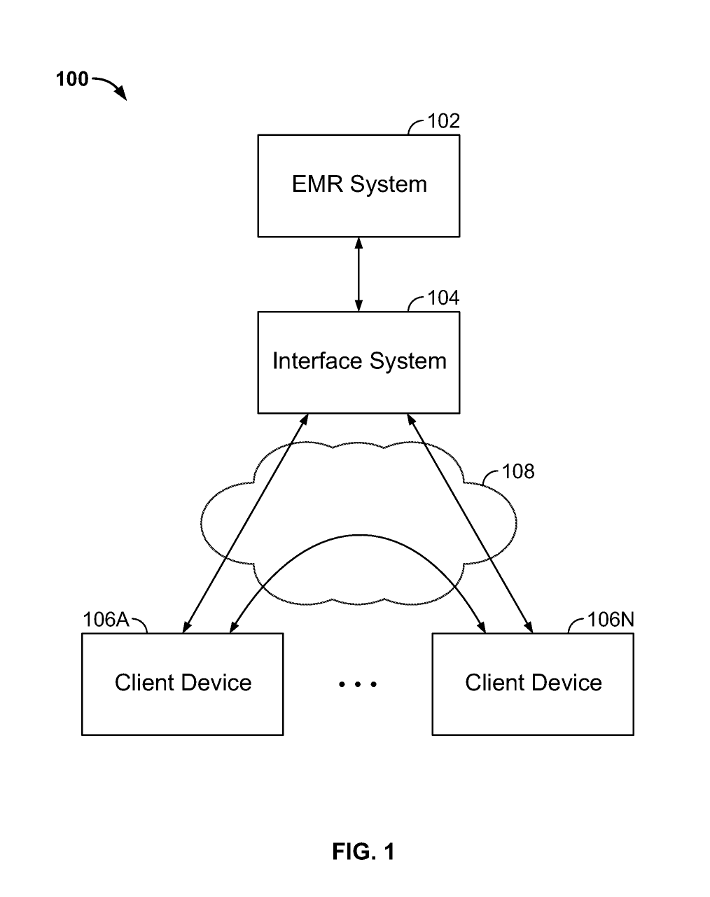

FIG. 1 is a block diagram illustrating an example system 100 for collecting measurements of physiological parameters of patients. As illustrated in the example of FIG. 1, the system 100 comprises an Electronic Medical Records (EMR) system 102, an interface system 104, a set of client devices 106A-106N (collectively, “client devices 106”), and a network 108.

The network 108 is an electronic communication network that facilitates communication between the client devices 106 and the between the client devices 106 and the interface system 104. An electronic communication network is a set of computing devices and links between the computing devices. The computing devices in the network use the links to enable communication among the computing devices in the network. The network 108 can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices. In various embodiments, the network 108 includes various types of links. For example, the network 108 can include wired and/or wireless links. Furthermore, in various embodiments, the network 108 is implemented at various scales. For example, the network 108 can be implemented as one or more local area networks (LANs), metropolitan area networks, subnets, wide area networks (such as the Internet), or can be implemented at another scale.

The EMR system 102 is a computing system that allows storage, retrieval, and manipulation of electronic medical records. As used herein, a computing system is a system of one or more computing devices. A computing device is a physical, tangible device that processes data. Example types of computing devices include personal computers, standalone server computers, blade server computers, mainframe computers, handheld computers, smart phones, special purpose computing devices, and other types of devices that process data.

Each client device in the set of client devices 106 is a computing device. The client devices 106 can provide various types of functionality. For example, the set of client devices 106 can include one or more physiological monitor devices (such as the physiological monitor device 200). In addition, the set of client devices 106 can include one or more desktop, laptop, or wall-mounted devices. Such wall-mounted devices can have similar functionality to the physiological monitor device 200 but are stationary instead of portable. In addition, the set of client devices 106 can include one or more physiological monitor devices. Such monitor devices can display representations of physiological parameters. A monitor device could, for example, be used by a clinician to monitor the physiological parameters of multiple patients at one time. Such monitor devices are typically not wall mounted.

The client devices 106 can communicate with each other through the network 108. In various embodiments, the client devices 106 can communicate various types of data with each other through the network 108. For example, in embodiments where the set of client devices 106 includes a set of physiological monitor devices and a monitor device, each of the physiological monitor devices can send data representing measurements of physiological parameters of patients to the monitor device. In this way, the monitor device can display representations of physiological parameters to a clinician.

The interface system 104 is a computing system that acts as an interface between the EMR system 102 and the client devices 106. In some embodiments, the interface system 104 is a CONNEX™ interface system from Welch Allyn of Skaneateles Falls, N.Y., although other interface systems can be used. Different EMR systems have different software interfaces.

For example, the EMR system used by two different hospitals can have two different software interfaces. The interface system 104 provides a single software interface to each of the client devices 106. The client devices 106 send requests to software interface provided by the interface system 104. When the interface system 104 receives a request from one of the client devices 106, the interface system 104 translates the request into a request that works with the software interface provided by the EMR system 102. The interface system 104 then provides the translated request to the software interface provided by the EMR system 102. When the interface system 104 receives a response from the EMR system 102, the interface system 104 translates the response from a format of the EMR system 102 to a system understood by the client devices 106. The interface system 104 then forwards the translated response to an appropriate one of the client devices 106.

The client devices 106 can send various types of data to the interface system 104 for storage in the EMR system 102 and can receive various types of data from the EMR system 102 through the interface system 104. For example, in some embodiments, the client devices 106 can send measurements of physiological parameters to the interface system 104 for storage in the EMR system 102. In another example, a monitor device can retrieve past measurements of physiological parameters of patients from the EMR system 102 through the interface system 104.

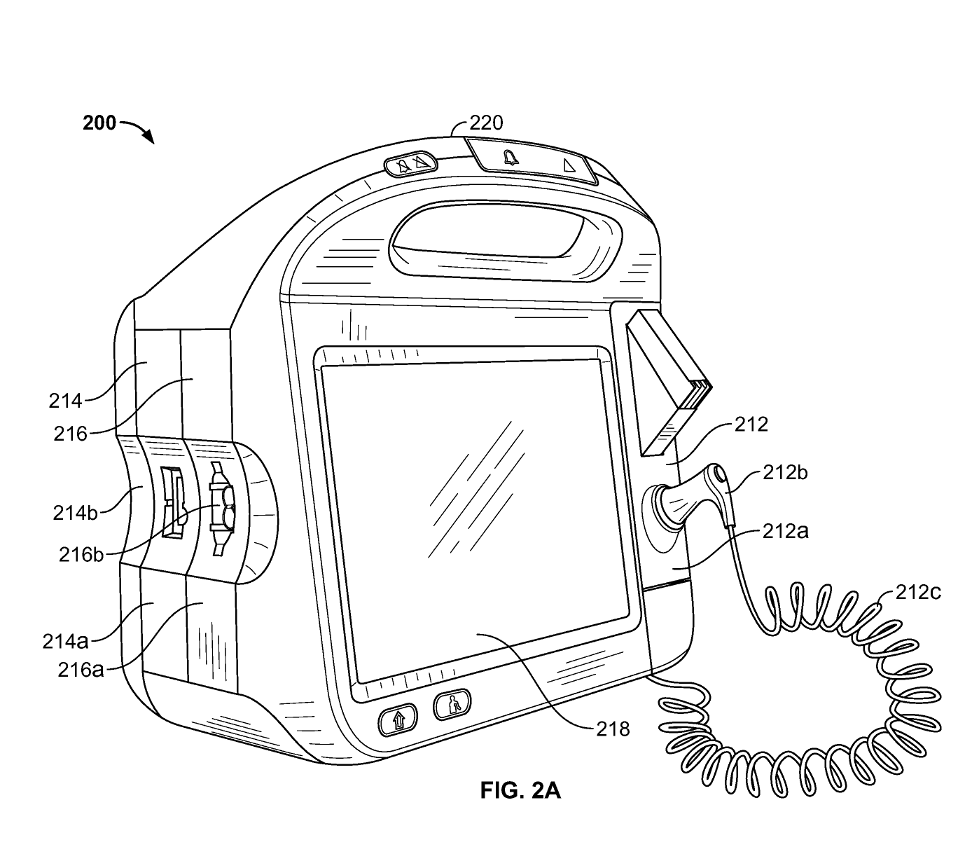

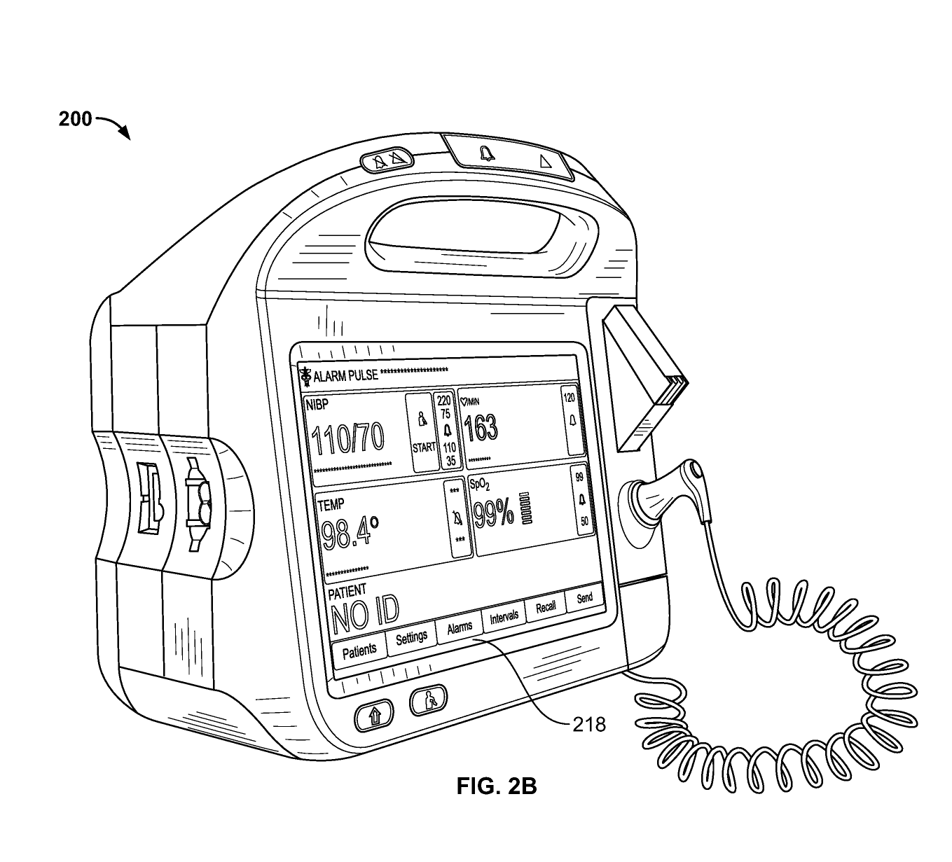

FIG. 2 illustrates a view of an example physiological monitor device 200. The physiological monitor device 200 is portable. The physiological monitor device 200 includes multiple health care equipment (HCE) modules. Each of the HCE modules is configured to measure one or more physiological parameters of a health-care recipient, also referred to herein as a patient.

A temperature measurement module 212 is accessible from the front side of the physiological monitor device 200. A SpO2 module 214 and a non-invasive blood pressure (NIBP) module 216 are accessible from a left hand side of the physiological monitor device 200. An upper handle portion 220 enables the physiological monitor device 200 to be carried by hand.

A front side of the physiological monitor device 200 includes a display screen 218 and an outer surface of the temperature measurement module 212. The temperature measurement module 212 is designed to measure the body temperature of a patient. As used in this document, a “module” is a combination of a physical module structure which typically resides within the physiological monitor device 200 and optional peripheral components (not shown) that typically attach to and reside outside of the physiological monitor device 200.

The temperature measurement module 212 includes a front panel 212a. The front panel 212a has an outer surface that is accessible from the front side of the physiological monitor device 200. The front panel 212a provides access to a wall (not shown) storing a removable probe (not shown), also referred to as a temperature probe, that is attached to a probe handle 212b. The probe and its attached probe handle 212b are tethered to the temperature measurement module 212 via an insulated conductor 212c. The probe is designed to make physical contact with a patient in order to sense a body temperature of the patient.

A left hand side of the physiological monitor device 200 includes an outer surface of the SpO2 module 214 and an outer surface of the NIBP module 216. The SpO2 module 214 is a HCE module designed to measure oxygen content within the blood of a patient. The NIBP module 216 is a HCE module designed to measure blood pressure of a patient.

As shown, the SpO2 module 214 includes a front panel 214a. The front panel 214a includes an outer surface that is accessible from the left side of the physiological monitor device 200. The front panel 214a includes a connector 214b that enables a connection between one or more peripheral SpO2 components (not shown) and a portion of the SpO2 module 214 residing inside the physiological monitor device 200. The peripheral SpO2 components reside external to the physiological monitor device 200. The peripheral SpO2 components are configured to interoperate with the SpO2 module 214 when connected to the SpO2 module 214 via the connector 214b. In some embodiments, the peripheral SpO2 components include a clip that attaches to an appendage of a patient, such as a finger. The clip is designed to detect and measure a pulse and an oxygen content of blood flowing within the patient.

As shown, the NIBP module 216 includes a front panel 216a having an outer surface that is accessible from the left side of the physiological monitor device 200. The front panel 216a includes a connector 216b that enables a connection between one or more peripheral NIBP components (not shown) and a portion of the NIBP module 216 residing inside the physiological monitor device 200. The peripheral NIBP components reside external to the physiological monitor device 200. The peripheral NIBP components are configured to interoperate with the NIBP module 216 when connected to the NIBP module 216 via the connector 216b. In some embodiments, the peripheral NIBP components include an inflatable cuff that attaches to an appendage of a patient, such as an upper arm of the patient. The inflatable cuff is designed to measure the systolic and diastolic blood pressure of the patient, the mean arterial pressure (MAP) of the patient, and the pulse rate of blood flowing within the patient.

The physiological monitor device 200 is able to operate within one or more workflows. A workflow is a series of one or more tasks that a user of the physiological monitor device 200 performs. When the physiological monitor device 200 operates within a workflow, the physiological monitor device 200 provides functionality suitable for assisting the user in performing the workflow. When the physiological monitor device 200 operates within different workflows, the physiological monitor device 200 provides different functionality.

When the physiological monitor device 200 is manufactured, the physiological monitor device 200 is configured to be able to operate within one or more workflows. After the physiological monitor device 200 is manufactured, the physiological monitor device 200 can be reconfigured to operate within one or more additional workflows. In this way, a user can adapt the physiological monitor device 200 for use in different workflows as needed.

In various embodiments, the physiological monitor device 200 operates within various workflows. For example, in some embodiments, the physiological monitor device 200 can operate within a monitoring workflow or a non-monitoring workflow. Example types of non-monitoring workflows include, but are not limited to, a spot check workflow and a triage workflow.

In example embodiments, the names for the workflows can be defined by the user. For example, the user can rename a “triage workflow” as “ED 3 North” or any other nomenclature as desired to provide more context to the user.

When the physiological monitor device 200 is operating within the monitoring workflow, the physiological monitor device 200 obtains a series of measurements of one or more physiological parameters of a single monitored patient over a period of time. In addition, the physiological monitor device 200 displays, on the display screen 218, a monitoring workflow home screen. The monitoring workflow home screen contains a representation of a physiological parameter of the monitored patient. The representation is based on at least one measurement in the series of measurements. A representation of a physiological parameter is a visible image conveying information about the physiological parameter.

For example, when the physiological monitor device 200 is operating within the monitoring workflow, the physiological monitor device 200 can obtain a blood pressure measurement of a single patient once every ten minutes for six hours. In this example, the physiological monitor device 200 displays a monitoring workflow home screen that contains a representation of the patient's blood pressure based on a most recent one of the temperature measurements. In this way, a user of the physiological monitor device 200 can monitor the status of the patient.

When the physiological monitor device 200 is operating within a non-monitoring workflow, the physiological monitor device 200 obtains a measurement of one or more physiological parameters from each patient in a series of patients. In addition, the physiological monitor device 200 displays a non-monitoring workflow home screen on the display screen 218. The non-monitoring workflow home screen contains a representation of the physiological parameter of a given patient in the series of patients. The representation is based on the measurement of the physiological parameter of the given patient.

In one example, when the physiological monitor device 200 is operating within a spot check workflow, the physiological monitor device 200 obtains blood pressure measurements from a series of previously-identified patients. In this other example, the physiological monitor device 200 displays a spot check workflow home screen containing a blood pressure measurement of a given patient in the series of previously-identified patients. In this way, a user of the physiological monitor device 200 can perform spot checks on the blood pressures of patients who have already been admitted to a hospital.

As used in this document, a patient is a previously identified patient when the physiological monitor device 200 stores information regarding the identity of the patient. In another example, when the physiological monitor device 200 is operating within a triage workflow, the physiological monitor device 200 can obtain a single blood pressure measurement from each patient in a series of unidentified patients as the patients arrive at a hospital. In this example, the physiological monitor device 200 displays a triage workflow home screen containing a representation of the patients' blood pressure based on the single blood pressure measurements of the patients. In this way, a user of the physiological monitor device 200 can perform triage on the series of unidentified patients as they arrive. As used in this document, a patient is an unidentified patient when the physiological monitor device 200 does not store information regarding the identity of the patient.

The monitoring workflow home screen is different than the non-monitoring workflow home screen. Further, as discussed below, the navigation options associated with the different workflows allows for efficient monitoring based on the environment in which the device is used. In various embodiments, the monitoring workflow home screen is different than the non-monitoring workflow home screen in various ways. For example, in some embodiments, the monitoring workflow home screen includes at least one user-selectable control that is not included in the non-monitoring workflow home screen. In other embodiments, a representation of a physiological parameter in the monitoring workflow home screen has a different size than a representation of the same physiological parameter in the non-monitoring workflow home screen.

FIG. 2B illustrates an example user interface displayed on the display screen 218 of FIG. 2A. The physiological monitor device 200 outputs and displays user interfaces discussed in this document on the display screen 218.

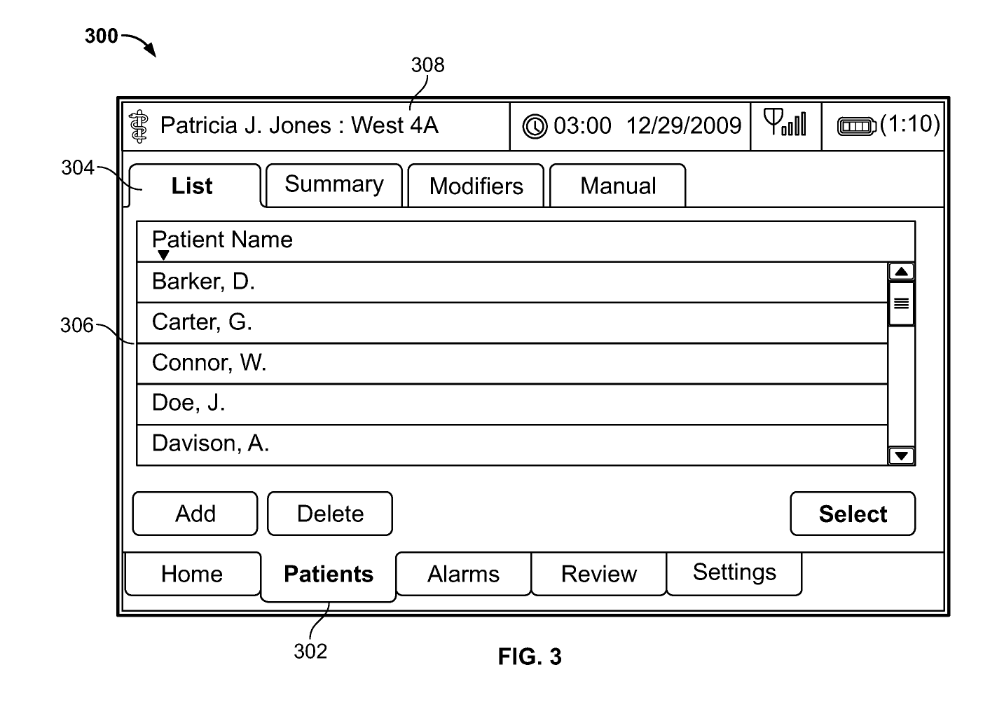

FIG. 3 illustrates an example patient list screen 300. The physiological monitor device 200 displays the patient list screen 300 when a Patients tab 302 and a List tab 304 are selected on the physiological monitor device 200. The example patient list screen 300 provides a list of patients 306 that are located in the same general area as the physiological monitor device 200, typically on the same floor. Each physiological monitor device has a unique device number associated with it. Each physiological monitor device also has a location field. In examples, the physiological monitor device number and PMP location field are pre-programmed into the interface system 104. For example, patient list screen 300 shows a location 308 as West 4A for physiological monitor device 200.

When the interface system 104 communicates with physiological monitor device 200 (through wired or wireless protocols), because the interface system 104 knows where physiological monitor device 200 is located, the interface system 104 provides a specific list of patients specific to the location of physiological monitor device 200. Typically, the list of patients is based on the floor on which physiological monitor device 200 is located. However, the list may also be based on a section of the floor, a room, etc. The interface system 104 obtains a list of hospital patients from EMR system 102. The interface system 104 creates the patient list on patient list screen 300 by filtering out patients not located in the area specified by the location field.

Using the example patient list screen 300, a clinician is able to walk into a room, select a patient from the list of patients displayed on patient list screen 300, take physiological measurement information for the patient and send the physiological measurement information to the interface system 104. The interface system 104 then sends the physiological measurement information to EMR system 102. Using patient list screen 300 to identify a patient obviates the need for including a barcode scanner in a patient monitoring device.

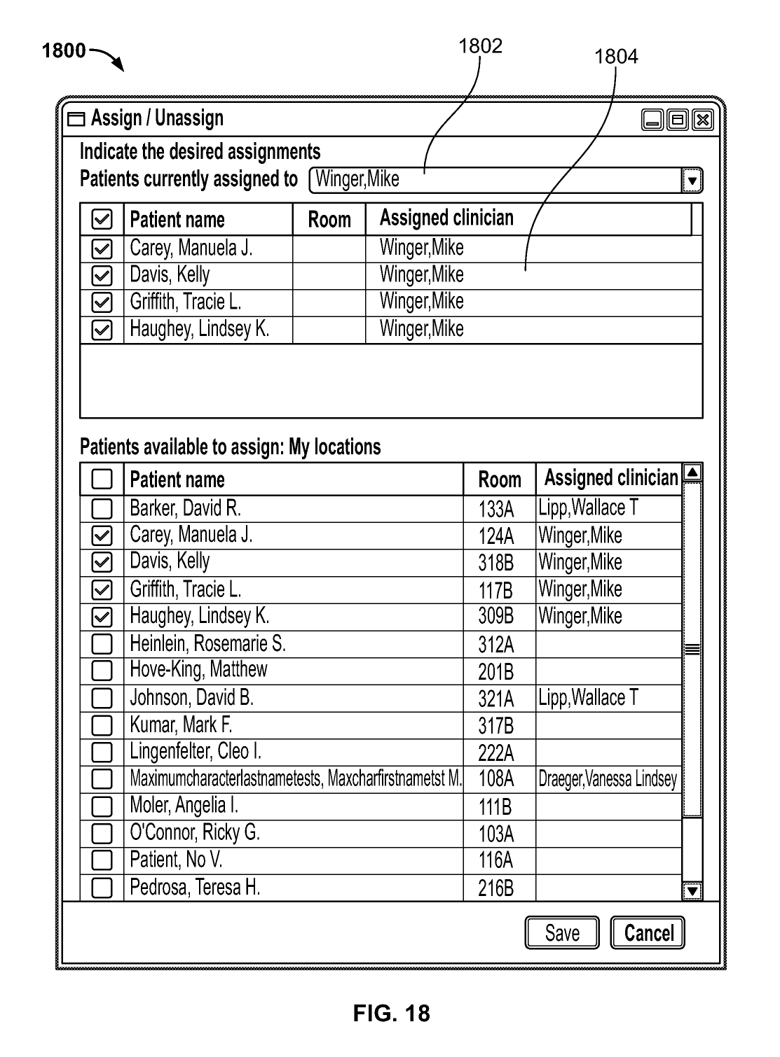

In alternative embodiments, the patient list can be selected based on other parameters. For example, as shown in FIG. 18, a patient list screen 1800 is provided. The patient list screen 1800 allows the clinician to input the clinician's name or ID, rather than a location, in the query box 1802. A patient list 1804 is returned including all of the patients assigned to or otherwise associated with that clinician. The clinician can then select the desired patient from the patient list 1804. In other examples, patient lists can be imported manually into the physiological monitor device 200.

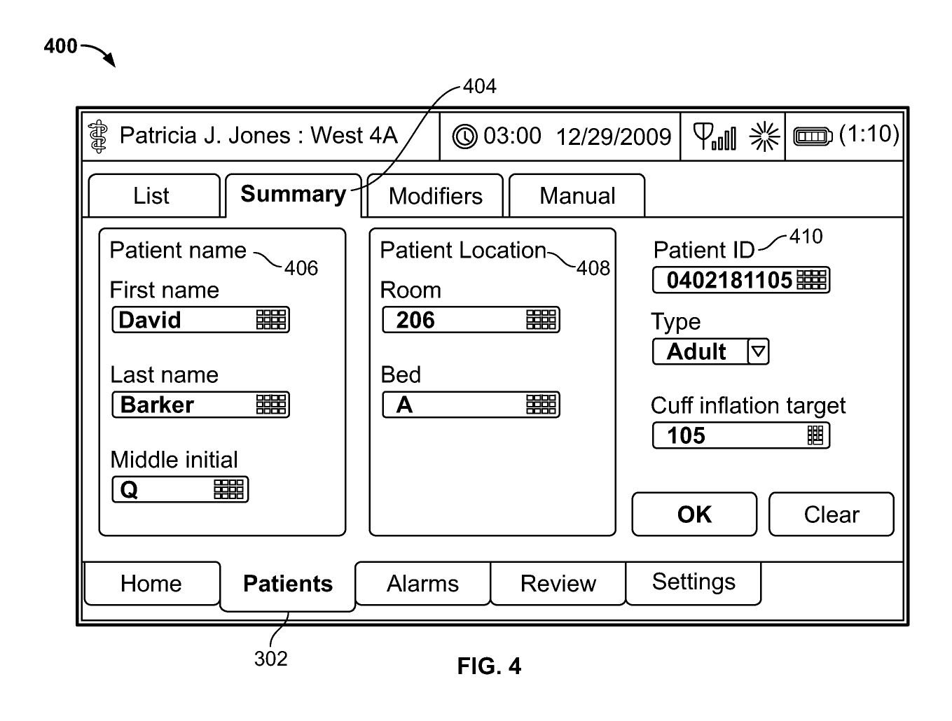

FIG. 4 illustrates an example patient summary screen 400. The physiological monitor device 200 displays the patient summary screen 400 when a Patients tab 302 and a Summary tab 404 are selected on the physiological monitor device 200. The patient summary screen 400 displays patient information including a patient name 406, patient location 408 and patient ID 410. The patient name 406 includes the first and last names of the patient and the middle initial of the patient. The patient location 408 includes a room number and bed number for the patient. The patient ID specifies a unique numerical identifier for the patient.

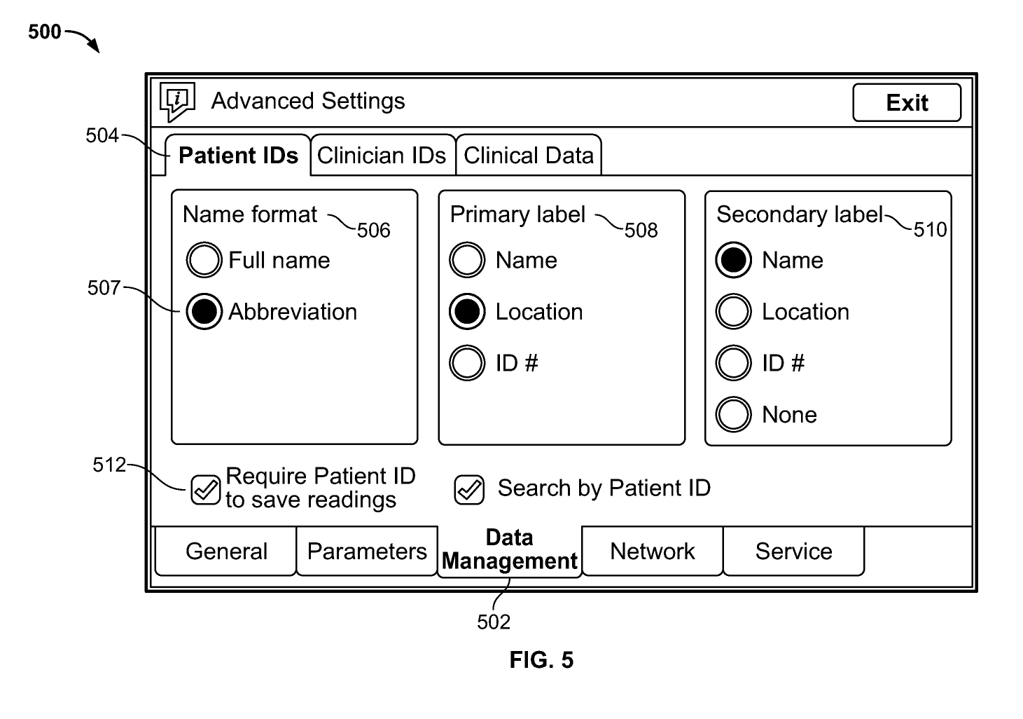

FIG. 5 illustrates an example data management screen 500. The physiological monitor device 200 displays the data management screen 500 when a Data Management tab 502 and a Patient IDs tab 504 are selected on the physiological monitor device 200. The data management screen 500 permits the selection of a format 506 for displaying a patient's name, the selection of a primary display label 508 and the selection of a secondary display label 508. When specifying a format 506 for displaying a patient's name on the physiological monitor device 200, either the patient's full name or an abbreviated name may be selected. A user selects the patient's full name or an abbreviated name by pressing a radio-type button associated with the patient's full name or an abbreviated name. As used herein, a radio-type button permits only one selection per functional grouping. Thus, for the functional grouping of a patient's name 506, only one of the patient's full name or an abbreviated name may be selected.

When specifying a primary display label on the physiological monitor device 200, either the patient's name, patient location or patient ID may be selected. The patient name, patient location and patient ID correspond to the patient name 406, the patient location 408 and the patient ID 410, respectively, of the patient summary screen 400. A user selects the patient name, the patient location or the patient ID by pressing a radio-type button associated with the patient name, the patient location or the patient ID.

When specifying a secondary display label on the physiological monitor device 200, either the patient name, patient location, patient ID or None may be selected. A selection of “None” indicates that only a primary display label is used. The patient name, patient location and patient ID used with the secondary display label are the same labels 406, 408 and 410, respectively used with the patient summary screen 400. A user selects the patient name, patient location, patient ID or none by pressing a radio-type button associated with the patient name, patient location, patient ID or none.

The patient summary screen 400 and the data management screen 500 provide for dual identification for a patient. In a hospital setting, proper identification of a patient is important. In many hospitals, when a patient is admitted to the hospital, the patient is given an identification wristband to wear. The identification wristband includes a barcode that corresponds to the patient ID. When a clinician scans the barcode with a scanner device, the patient ID can be displayed on a display device, for example on physiological monitor device 200. However, the barcode is typically a long alphanumeric number, typically 25 or 30 characters in length. When a clinician views the barcode on the display device, it is often difficult to verify the identity of the patient simply by viewing the patient ID. Therefore, the physiological monitor device 200 permits the display of a dual ID, typically the patient ID and either the patient name or the patient location.

In examples, when a clinician scans the barcode on an identification wristband, the scanned barcode is sent to the interface system 104, which looks up the scanned barcode in its database. The database is continuously updated based on information received from EMR system 102. The EMR system 102 uses an admit and discharge transfer (ADT) functionality of the EMR system to communicate patient information to system 104.

When the interface system 104 finds the matching information in the database, the interface system 104 sends the received patient name and patient location to the physiological monitor device 200. The physiological monitor device 200 uses the information selected in the data management screen 500 to determine the patient identification to be displayed on the physiological monitor device 200. For example, if the primary label 508 is the patient ID (e.g., a number or series of characters) and the secondary label 510 is the patient location, the patient ID and the patient location are displayed on physiological monitor device 200. Similarly, if the primary label 508 is the patient ID and the secondary label 510 is the patient name, the patient ID and the patient name are displayed on physiological monitor device 510.

In other examples, other individuals, such as clinicians, can also be identified using barcodes. For example, the clinician can scan a barcode associated with the clinician to login or otherwise identify the clinician for the physiological monitor device 200.

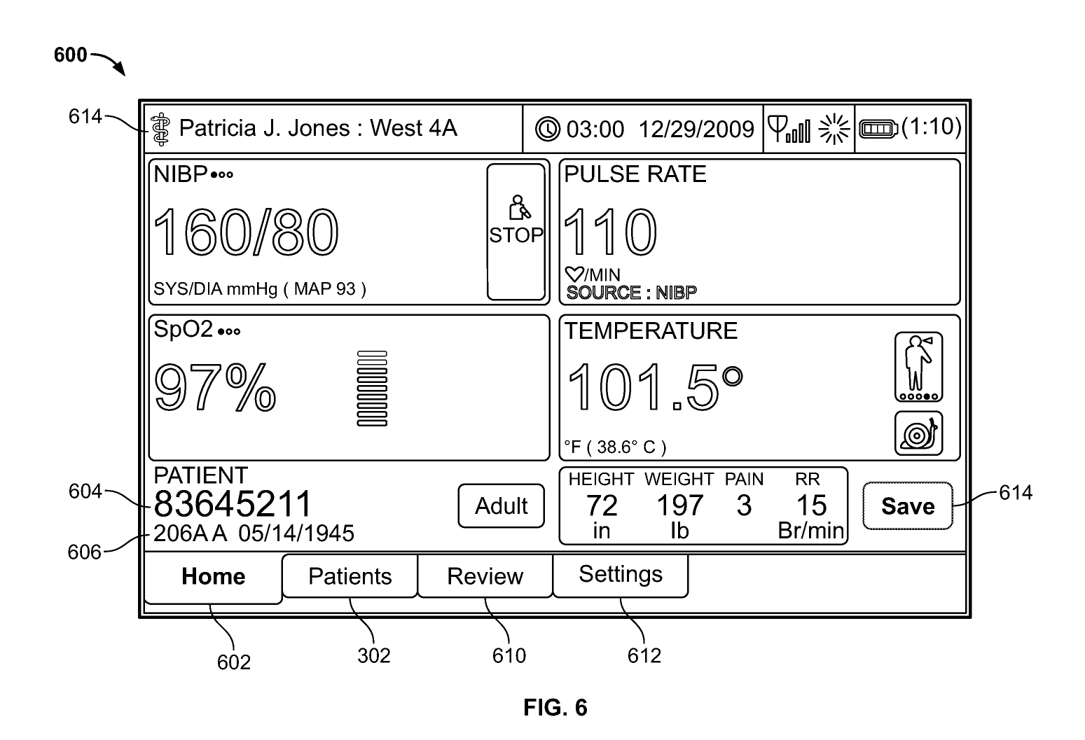

FIG. 6 illustrates an example home screen 600. The physiological monitor device 200 displays the home screen 600 when a Home tab 602 is selected on the physiological monitor device 200.

Patient identification information is displayed at the lower left portion of the example home screen 600. In the example home screen 600, the patient's ID 602 is displayed above a location code 604 for the patient. In the example home screen 600, the patient ID is displayed above the location code 604 and is displayed in a larger font than the location code 604. In example home screen 600, the patent ID is associated with the primary label 508 and the location code 604 is associated with the secondary label 510. The patient's ID 602 is associated with the primary label 508 and the location code 604 is associated with the secondary label 510 via a selection made on the example data management screen 500. The example location code 604 is displayed as 206A, corresponding to room 206, bed A as entered on patient summary screen 400. In examples, if the patient name 406 is selected as the secondary label 510 instead of location code 604, the patient name 406 is displayed below the patient ID 602 on home screen 600.

FIGS. 3, 4, 7 and 8 illustrate how the physiological monitor device 200 can be used for integrated patient management. Integration patient management permits a plurality of information to be entered and organized for a patient.

FIG. 3 illustrates an example patient list screen 300. When a patient 306, for example Barker, D, is selected from patient list screen 300, an example patient summary screen 400 (see FIG. 4) is displayed for the selected patient David Barker. As discussed, the patient summary screen 400 permits entry and display of the patient name 406, the patient location 408 and the patient ID 410.

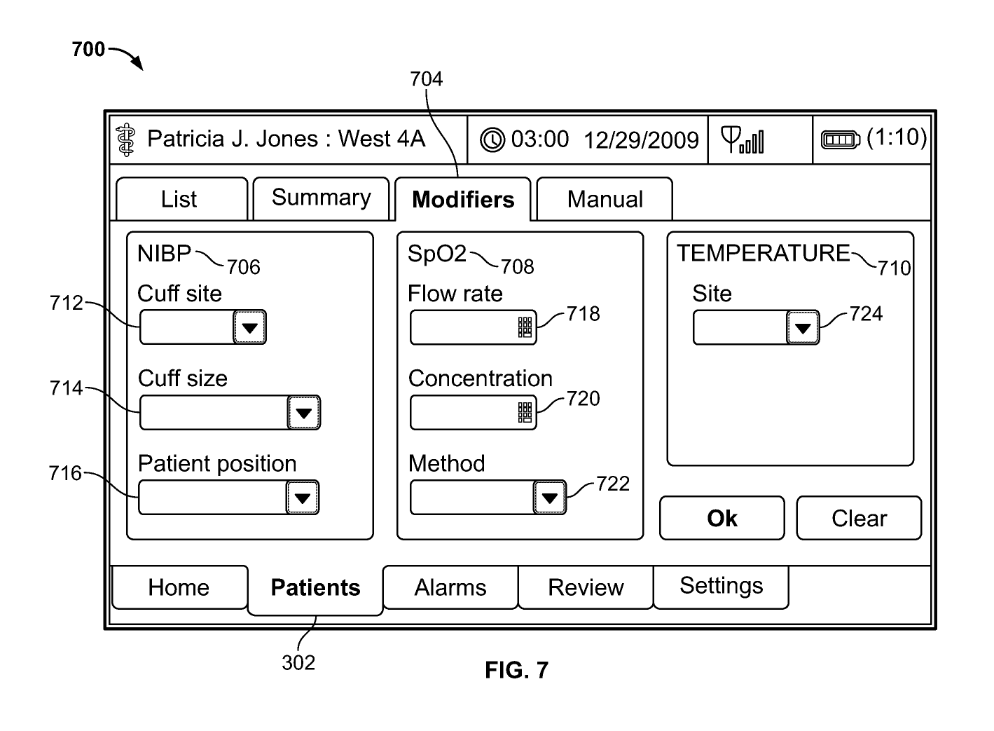

FIG. 7 illustrates an example patient modifier screen 700. The physiological monitor device 200 displays the patient modifier screen 700 when a Patients tab 302 and a Modifiers tab 704 are selected on the physiological monitor device 200. The patient modifier screen 700 permits a clinician to capture additional information associated with patient readings for NIBP, SPO2 and temperature, sometimes referred to as modifiers and qualifiers. The patient monitor screen 700 includes a NIBP area 706, a SPO₂ area 708 and a temperature area 710.

The example NIBP area 706 permits a clinician to enter information regarding the location of a blood pressure cuff on the patient, the size of the blood pressure cuff and the position of the patient during the NIBP procedure. The location of the cuff is entered via a scroll-down list box 712 for cuff site. The scroll-down list box 712 displays a plurality of cuff site locations, for example the left arm, the right arm, the left leg, etc. from which a cuff site can be selected. The cuff size is entered via a scroll-down list box 714 for cuff size. One of a plurality of cuff sizes, for example infant, child, adult, large adult, etc., may be selected. The patient position is entered via a scroll-down list box 716 for patient position. A patient position, such as lying, sitting or standing, may be selected via scroll-down list box 716.

The example SPO2 area 708 permits a clinician to enter information regarding flow rate, concentration and method. The flow rate corresponds to the flow rate of oxygen used with a patient when obtaining a SPO2 reading. The concentration corresponds to the concentration of oxygen used with a patient when obtaining a SPO2 reading. The method corresponds to the method used to deliver oxygen to the patient when obtaining a SPO2 reading. The flow rate is entered via edit box 718. Typical flow rates range from 1 to 20 liters/minute. The concentration is entered via edit box 720. Typical concentrations range from 21% to 100%. The method for delivering oxygen is entered via scroll-down list box 722. Example methods displayed via the scroll-down list box 722 include a face tent, mask and ventilator. Are methods are possible.

The example temperature area 710 permits a clinician to enter information regarding a type of temperature reading mechanism used to obtain the temperature of a patient. The temperature reading mechanism is entered via scroll-down list box 724. Example temperature reading mechanisms displayed via the scroll-down list box 724 include axillary, oral and rectal. Other temperature reading mechanisms are possible.

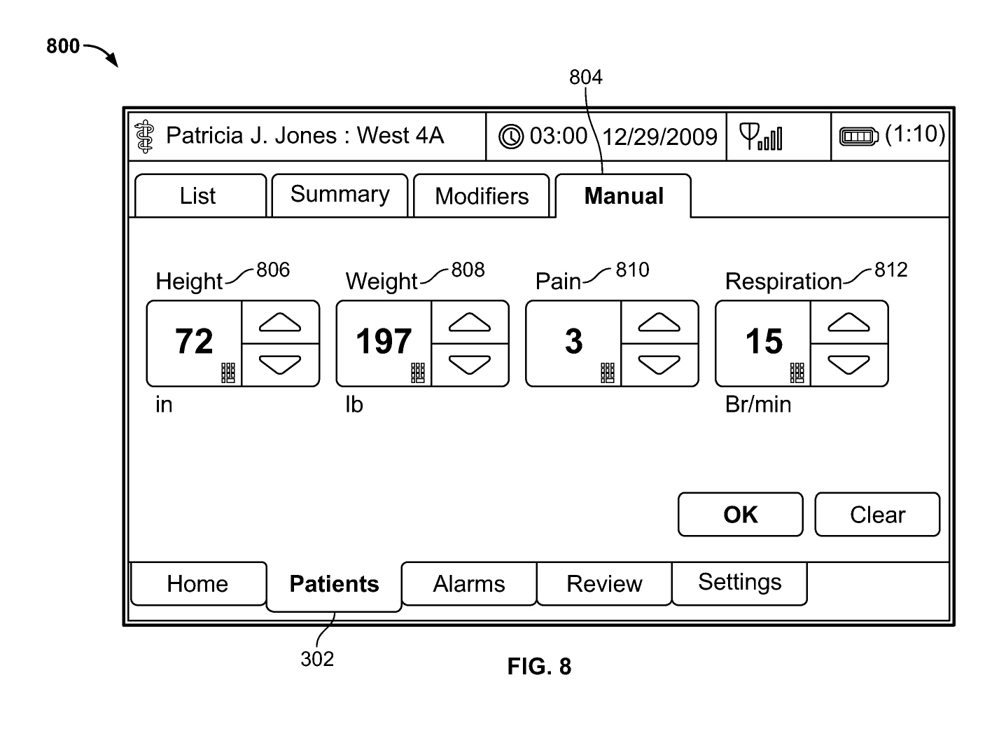

FIG. 8 illustrates an example screen 800 for entering patient information. The physiological monitor device 200 displays screen 800 when a Patients tab 302 and a Manual tab 804 are selected on the physiological monitor device 200. Screen 800 permits information including a patient's height, weight, pain level and respiration rate to be manually entered into the physiological monitor device 200. The patient's height is entered via edit box 806. The patient's weight is entered via edit box 808. The patient's pain level, typically a value from 0 to 10 where 0 indicates no pain and 10 indicates the highest level of pain, is entered via edit box 810. The patient's respiration rate in breaths per minute, typically in a range from 0 to 99, is entered via edit box 812.

In example embodiments, this manual information that is entered on the physiological monitor device 200 is communicated to the interface system 104. The interface system 104 is configured to map the collected information so that the information can be sent to and stored in the EMR system 102. For example, if the physiological monitor device 200 is programmed to store the patient's weight in pounds, but the EMR system 102 stores the patient's weight in kilograms, the interface system 104 is programmed to automatically convert the reading before sending the reading from the physiological monitor device 200 to the EMR system 102 for storage. Similarly, if the physiological monitor device 200 stores the position of a blood pressure cuff as “left arm,” and the ERM 102 stores the parameter as “LARM,” the interface system 104 is programmed to make the necessary mapping.

The physiological monitor device 200 also includes a plurality of features directed to protecting patient data. Included among these features is abbreviations to protect identifiable information, manual or automatic timed partial screen blackouts, symbols to protect identifiable clinician and patient information, acknowledgment of a successful transmission of the patient record to EMR system 102, automatic deletion of a patient record after a successful transmission of the patient record to EMR system 102, automatic deletion of old patient data greater than 24 hours, configurable rules to require patient and clinician ID, automatic send of confirmed patient data, automatic forced review of unconfirmed patient data and automatic filtering of device memory based on a patient ID.

When patient names are displayed on the physiological monitor device 200, the privacy of the patient is exposed to any person who is able to view the physiological monitor device 200. For example, the patient list screen 400 illustrated in FIG. 4 displays a list of patients on a floor. A visitor in the room of one of the patients is able to see the list of all the other patients on the floor. To ensure the privacy of patients, some facilities abbreviate names, so that the identity of these patients is not easily discernable.

As discussed, the example data management screen 500, illustrated in FIG. 5, permits a user or an administrator at the caregiver location to specify that a particular patient's name is abbreviated. For example, the format and data provided can be configured per a hospital's protocol, so that when “abbreviation” is selected, all patients in the device are formatted based on this rule. The user specifies that a patient's name is to be abbreviated by pressing the Abbreviation radio-type button 507 in the Name Format area 506 of the data management screen 500. In addition, a patient may choose not to have their name displayed at all on the example home screen 600 illustrated in FIG. 6. To configure the physiological monitor device 200 so that a patient's name is not displayed on the home screen 600, a user can select a primary label 508 other than a name, for example a patient ID or a location, and a user can either select a secondary label 510 other than a name, for example a patient ID or a location, or select None for secondary label 510. When None is selected for a secondary label 510, a secondary label is not displayed on the home screen 500.

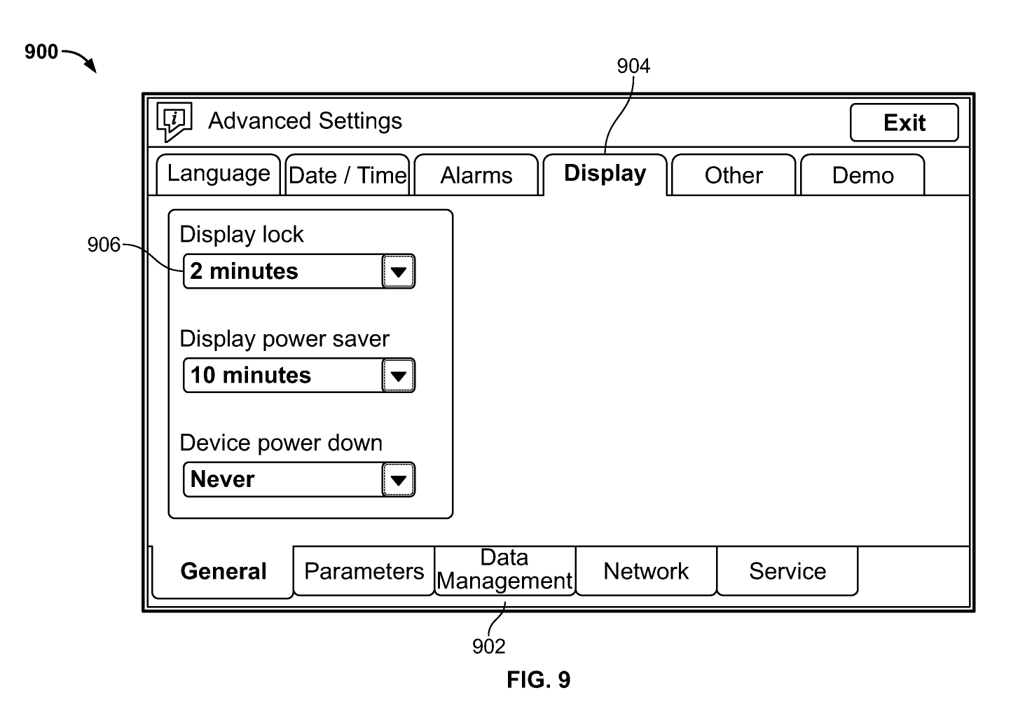

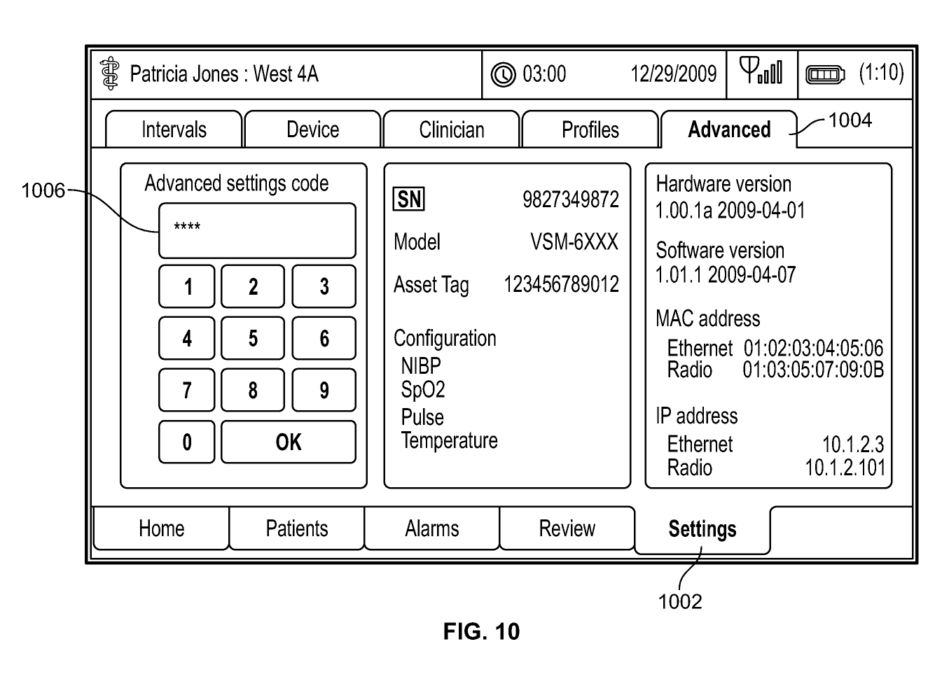

FIG. 9 illustrates an example advanced settings screen 900 for setting an automatic display lockout. The physiological monitor device 200 displays advanced settings screen 900 when advance settings are authorized and when a General tab 902 and a Display tab 904 are selected on the physiological monitor device 200. Advanced settings are authorized when a password is entered into physiological monitor device 200 that permits user access to advanced settings. The user enters the password into physiological monitor device 200 when the Settings tab 1002 and an Advanced tab 1004 are selected on the physiological monitor device 200 (FIG. 10). The user password is entered into advanced settings code area 1006.

The automatic display lockout is selected on the advanced screen 900 via a scroll-down list box 906. The scroll-down list box 906 displays time values of inactivity after which a touch screen, for example home screen 600, of physiological monitor device 200 is locked. Example time periods of inactivity that may be selected include 2 minutes, 5 minutes, 10 minutes, 15 minutes, 20 minutes and 30 minutes. Other time periods of inactivity are possible.

When a selected time period of inactivity expires, in addition to the home screen 600 being locked, portions of the home screen 600 are blacked out, hiding sensitive patient and clinician information. In examples, tabs 602, 302, 610 and 612 are blacked out, the top portion 614 of home screen 600 is blacked out and patient information such as the patient ID 604 and the patient name and location are blacked out. The only information displayed on home screen 600 is the active patient physiological measurements being monitored.

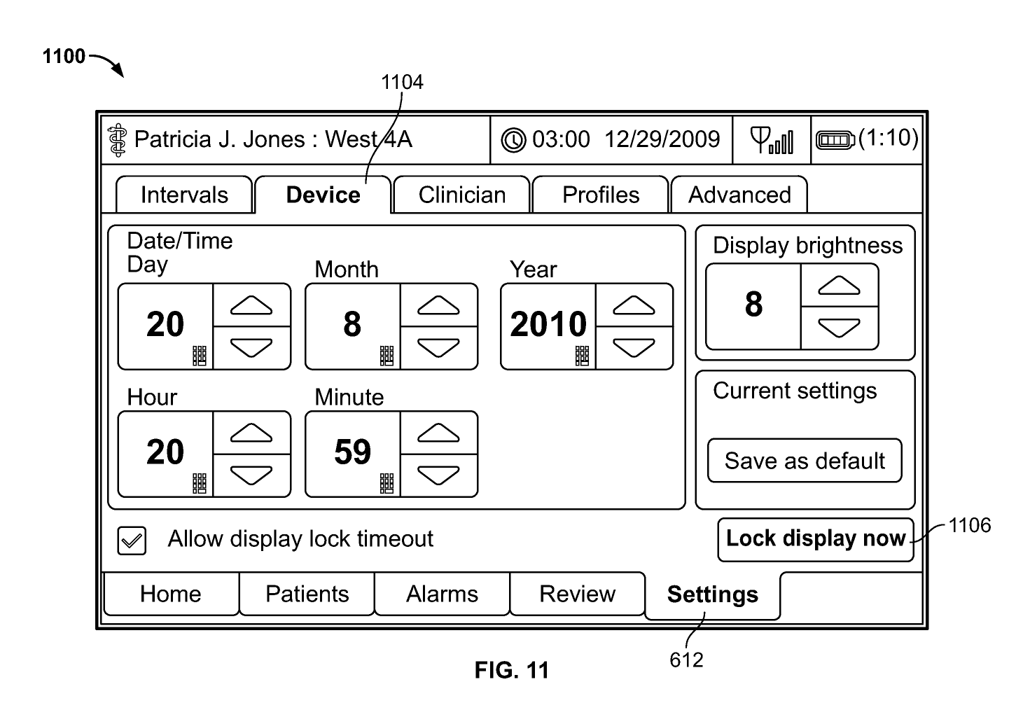

In addition, the home screen 600 can be locked out manually. FIG. 11 illustrates an example screen 1100 from which screens on the physiological monitor device 200, including home screen 600, can be locked out manually. The example screen 1100 is displayed when the Settings tab 612 and the Device tab 1104 are selected on the physiological monitor device 200. The screen 1100 includes a Lock Display Now button 1106. When the Lock Display Now button 1106 is pressed, the home screen 600 is blacked out in the same manner as that described for automatic display lockout. In examples, tabs 602, 302, 610 and 612 are blacked out, the top portion 614 of home screen 600 is blacked out and patient information such as the patient ID 604 and the patient name and location are blacked out. The only information displayed on home screen 600 is the active patient physiological measurements being monitored.

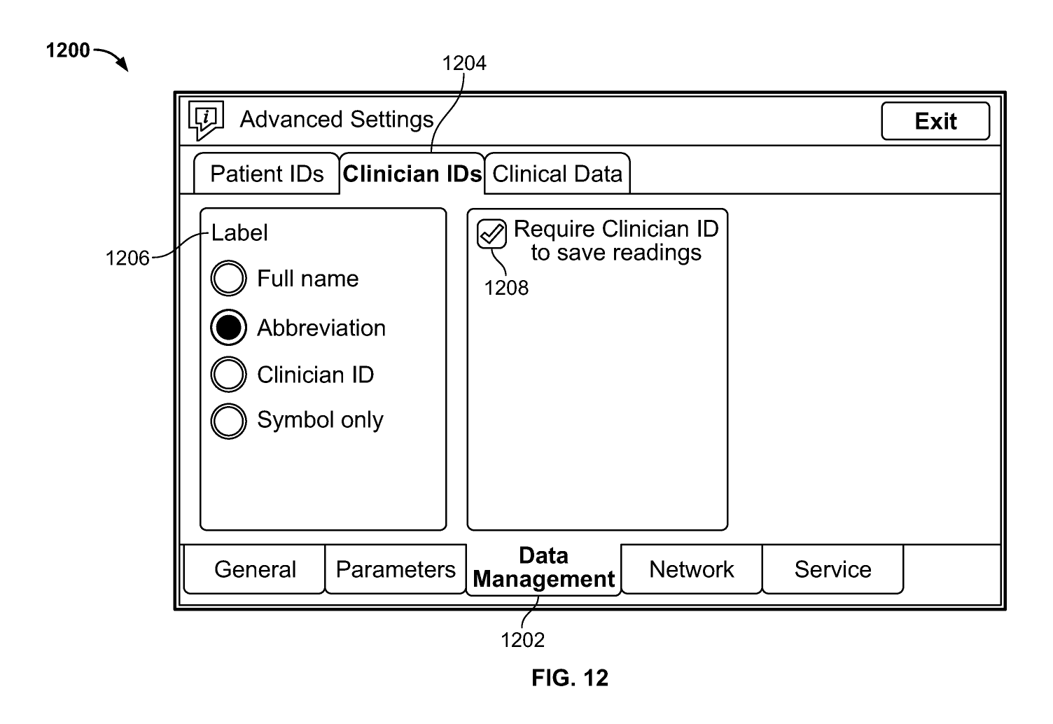

The physiological monitor device 200 permits a clinician to be identified by a symbol as a means of protecting the identity of the clinician. FIG. 12 illustrates an example advanced settings screen 1200 for selecting how clinician information is to be displayed on the physiological monitor device 200. The example screen 1200 is displayed when the Data Management tab 1202 and the Clinician IDs tab 1204 are selected on an advanced settings screen. An advanced setting screen is selected as illustrated in FIG. 10 and as discussed.

The example advanced settings screen 1200 includes a label section 1206 that presents alternate ways of identifying a clinician. The clinician may be identified by the full name of the clinician, by an abbreviation of the full name, by the clinician ID or by a symbol. The way in which the clinician is identified is selected via a radio-type button associated with each of full name, abbreviation, clinician ID and symbol only.

When symbol only is selected, the clinician is identified only by a symbol, for example the symbol 614 at the top left of the device status area of FIG. 6. The symbol 614 is displayed instead of the clinician name and location shown in FIG. 6. The symbol protects the identity of the clinician. The symbol indicates that a clinician is assigned to the patient but doesn't disclose the identity of the clinician. If additional information about the clinician is needed, a user of physiological monitor device 200 needs to know how to navigate the screens in the physiological monitor device 200 to locate a screen in which clinician identification is entered.

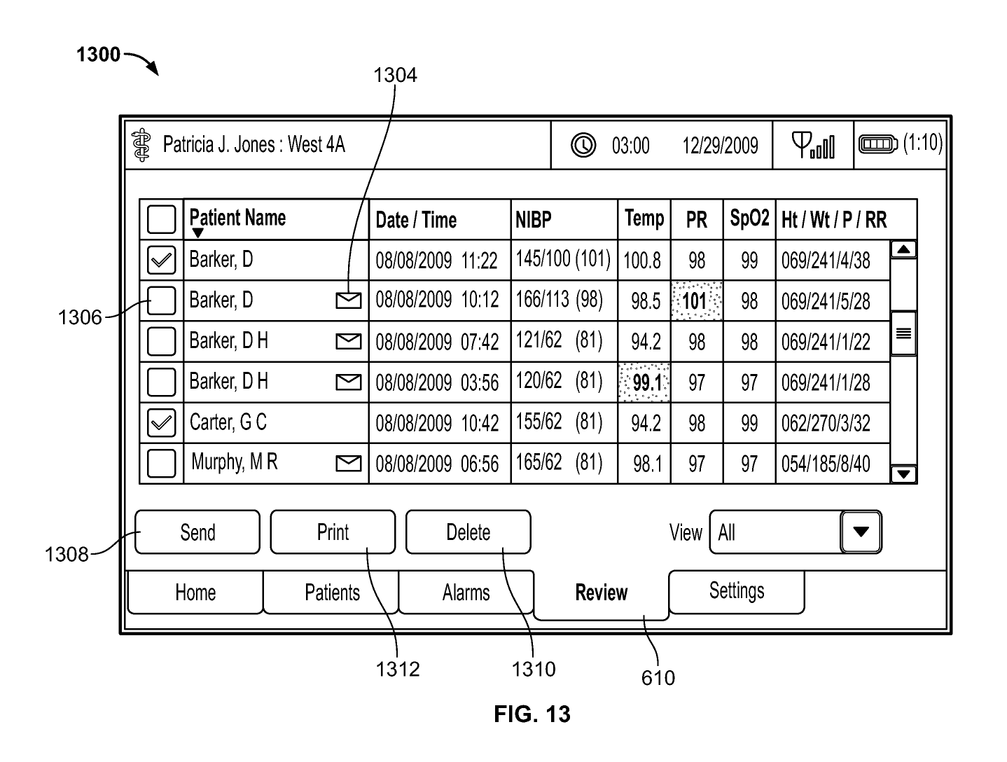

As an aid for a clinician to know when to delete physiological measurement records from the physiological monitor device 200, the physiological monitor device 200 displays an acknowledgment when physiological measurement records are successfully sent to EMR system 102. FIG. 13 shows an example review screen 1300. The example review screen 1300 is displayed when the Review tab 1302 is selected on the example home screen 600. The review screen 1300 displays a listing of physiological measurement records taken for a plurality of patients. As shown in the review screen 1300, a physiological measurement record comprises a patient name, time stamp, non-invasive blood pressure reading (NIBP), pulse reading (PR), SPO2 reading, height, weight, pain level and respiration rate. For example, the review screen 1300 displays physiological measurement readings for patient 1306, D. Barker, taken on Aug. 18, 2008 at 10:12. When a clinician decides to send these physiological measurement readings for D. Barker to the EMR system 102 (through the interface system 104), the clinician presses the send button 1308.

When the EMR system 102 acknowledges receipt of the physiological measurement records sent, the EMR system 102 sends an acknowledgment to the interface system 104, which forwards the acknowledgment to the PMP system 200. When the PMP system 200 receives the acknowledgment, the PMP system 200 displays a symbol, typically an envelope, to indicate the acknowledgment. For example, envelope 1304 is displayed to acknowledge that the physiological measurement records sent to the EMR system 102 for D. Barker has been received. The clinician can now delete the displayed physiological measurement records. The clinician deletes a physiological measurement record by selecting the row 1306 on which the physiological measurement record is displayed and by pressing the delete button 1310.

Alternatively, physiological measurement records sent to the EMR system 102 can be automatically deleted. FIG. 13 illustrates an example advanced settings screen 1400 that is used for configuring the PMP system 200 to automatically delete physiological measurement records after a successful send to the EMR system 102. The example advanced settings screen 1400 is displayed when the Data Management tab 1402 and the Clinical Data tab 1404 are selected on an advanced settings screen of the PMP system 200. The advanced settings screen includes a checkbox 1406 for Delete readings after a successful send. When the checkbox 1406 is checked, physiological measurement records that are successfully sent to the EMR system 102 are automatically deleted when the EMR system 102 acknowledges that the physiological measurement records have been successfully received. Instead of displaying envelope 1304, the physiological measurement record 1306 for D. Barker is deleted from the review screen 1300.

In addition, to manual and automatic deletion of patient readings as discussed, the PMP system 200 automatically deletes all physiological measurement records displayed on review screen 1300 after 24 hours. For example, when a row of physiological measurement readings is displayed on review screen 1300, the row of physiological measurement readings is automatically deleted 24 hours after it is displayed. Deleting physiological measurement records improves security since these records are not available for viewing by others. In the example shown, each record/row is independent. In other words, if Reading A is taken at 1:00 PM and reading B is taken at 1:05 PM, reading A will be deleted the next day at 1:00 PM and reading B at 1:05 PM. Other configurations are possible.

Physiological measurement records may also be printed from the review screen 1300. When the example print button 1312 is pressed, all selected physiological measurement records are printed. A separate report is printed for each patient selected. Each report has a header identifying the name of the patient.

Another data protection feature of the physiological monitor device 200 is to configure the physiological monitor device 200 to require a patient and clinician ID to be entered before physiological measurement records are sent to the EMR system 102. Certain physiological measurement records require a patient ID and clinician ID to be included when the records are sent to the EMR system 102. Configuring the physiological monitor device 200 to require the patient ID and clinician ID to be entered causes the PMP 200 device 200 to prompt the user when sending physiological measurement records that require a patient ID and clinician ID. Providing the patient ID and clinician ID when needed minimizes the chances that when physiological measurement records are sent they will not be rejected by EMR system 102. Configuring the physiological monitor device 200 to require a patient ID and clinician ID also ensures that unauthorized information is not entered into the physiological monitor device 200.

FIG. 5 illustrates an advanced settings screen 500 that is used to configure the physiological monitor device 200 to require a patient ID to save readings. The advanced settings screen 500 is displayed when the Data Management tab 502 and when the Patient IDs tab 504 is selected on an advanced settings screen. When checkbox 512 is checked on the advanced settings screen 500, the physiological monitor device is configured to require a patient ID before physiological measurement readings can be saved on the physiological monitor device 200.

FIG. 12 illustrates an advanced settings screen 1200 that is used to configure the physiological monitor device 200 to require a clinician ID to save readings. The advanced settings screen 1200 is displayed when the Data Management tab 1202 and when the Clinician IDs tab 1204 is selected on an advanced settings screen. When checkbox 1208 is checked on the advanced settings screen 1200, the physiological monitor device is configured to require a clinician ID before physiological measurement readings can be saved on the physiological monitor device 200.

As an additional data protection feature, the physiological monitor device 200 automatically filters the memory of the physiological monitor device 200 based on patient ID. When a patient ID, for example patient ID 604, is displayed on the example home screen 600, only data for the patient associated with the patient is displayed on the physiological monitor device 200. For example, when a patient ID is displayed on the example home screen 600, the physiological measurement readings of only that patient are displayed on the example review screen 1300 of FIG. 13. This protects the privacy of other patient's data and organizes the review screen 1300 to present pertinent data to the clinician. In addition, users can sort the order of the patients' data by parameters such as date/time in ascending or descending order.

The physiological monitor device 200 also forces physiological measurement records to be sent to the EMR system 102 from the example review screen 1300 unless the physiological monitor device 200 is configured to automatically send physiological measurement records to the EMR system 102 on a manual save. A manual save occurs when the example save button 614 is pressed on the example home screen 600.

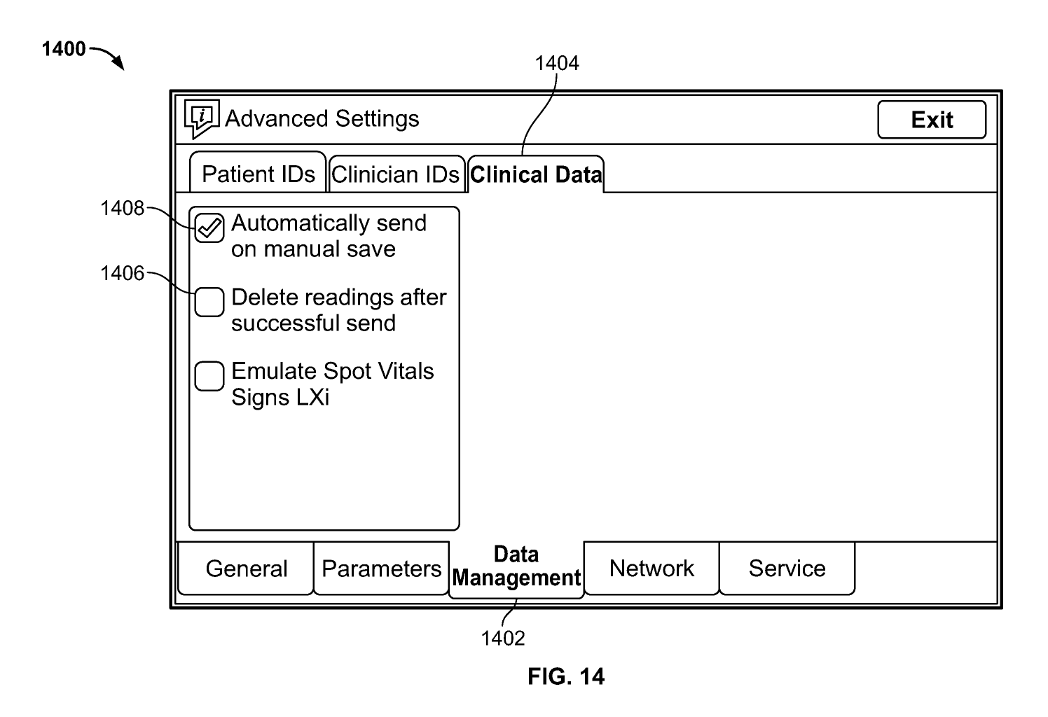

FIG. 14 illustrates an advanced settings screen 1400 that is used to configure the physiological monitor device 200 to automatically send physiological measurement records to the EMR system 102 (through the interface system 104) on a manual save. The example advanced settings screen 1400 is displayed when the Data Management tab 1402 and the Clinical Data tab 1404 are selected on an advanced screen. When checkbox 1408 is checked, the physiological monitor device 200 is configured to automatically send physiological measurement records to the EMR system 102 on a manual save. When checkbox 1404 is checked, each time the save button 614 is pressed on the example home screen 600, physiological measurement records for the patient identified via labels 604 and 606 are sent automatically sent to the EMR system 102.

When checkbox 1404 is not selected, physiological measurement records are only sent to EMR system 102 from the example review screen 1300. Typically, the checkbox 1404 is not checked when physiological measurement readings are taken for a patient when a clinician is not in the room with the patient when the physiological measurement readings are taken. The clinician will typically want to review the readings to make sure the readings are accurate before sending the readings to the EMR system 102. Using review screen 1300, the clinician checks each row of readings the clinician wants to send. When the clinician presses the example send button 1308, the readings of each checked row are sent to the EMR system 102.

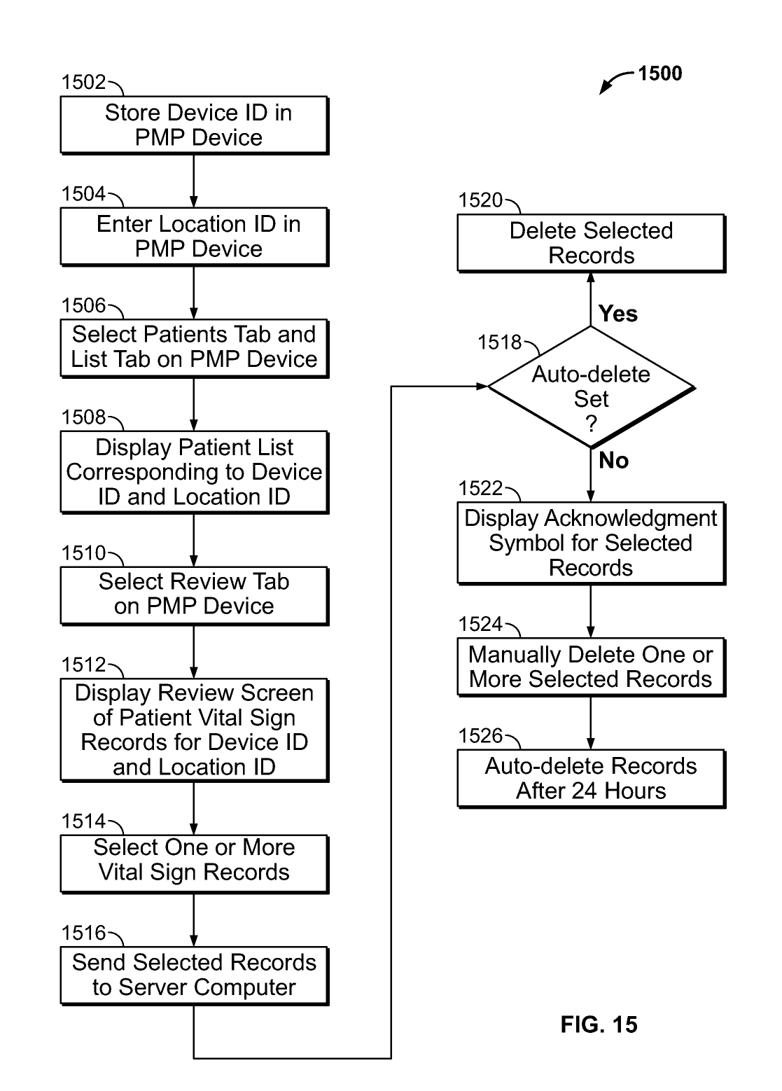

FIG. 15 is a flowchart illustrating an example method 1500 for displaying a list of patients on physiological monitor device 200, reviewing physiological measurement records for these patients and sending one or more records to the EMR system 102. At operation 1502, a device ID is stored in the physiological monitor device 200. The device ID, typically a serial number for the physiological monitor device, is a unique number that identifies the physiological monitor device 200. The device ID is stored in system memory in the physiological monitor device 200.

At operation 1504, a location ID is entered into the physiological monitor device 200 and stored in system memory of the physiological monitor device 200. The location ID in entered into physiological monitor device 200 via an advanced settings screen of the physiological monitor device 200. The location ID specifies a location for the physiological monitor device 200, typically a room number, a bed or a floor in a medical facility.

At operation 1506, a user, typically a clinician, selects a patient tab and a list tab, for example patient tab 302 and list tab 304 on the physiological monitor device 200. As a result of selecting the Patients tab and the List tab, at operation 1508, the physiological monitor device 200 displays a patient list screen, for example patient list screen 300 on the physiological monitor device 200.

The patient list screen 300 displays a list of patients based on the location of the physiological monitor device 200. When the Patients tab and the List tab are selected, the physiological monitor device 200 sends the location ID and the device ID to the interface system 104. The interface system 104 is typically preprogrammed with the location ID and the device ID of the physiological monitor device 200. The interface system 104 also stores the names of all patients entered into the EMR system 102. When the interface system 104 receives the location ID and device ID from the physiological monitor device 200, the interface system 104 filters the list of all patients and sends a list of patients corresponding to the location ID and the device ID to the physiological monitor device 200.

At operation 1510, a review tab, for example Review tab 610 is selected on the PMP system 200. As a result of selecting the review tab, at operation 1512 a review screen, for example review screen 1300, is displayed on the physiological monitor device 200. The review screen 1300 displays a list of physiological measurement records for patients at the location specified by the location ID, for example patients on a floor or patients in a room specified by the location ID.

At operation 1514, a user, typically a clinician, selects one or more the physiological measurement records. The user selects a physiological measurement record by clicking on a checkbox, for example checkbox 1306, adjacent to a patient name.

At operation 1516, the user clicks on send button 1308 to send the selected physiological measurement records to the EMR system 102. The EMR system 102 is a server computer. The selected physiological measurement records are sent directly to interface system 104, typically the interface system 104, and the interface system 104 forwards the physiological measurement records to the EMR system 102.

At operation 1518, a determination is made as to whether the physiological monitor device 200 is configured for auto-delete of successfully sent physiological measurement records. The physiological monitor device 200 is configured for auto-delete of successfully sent physiological measurement records when check box 1406, delete readings after successful send, is checked on the example advanced settings screen 1400.

When a determination is made at operation 1518 that the physiological monitor device 200 is configured for auto-delete of successfully sent physiological measurement records, at operation 1520, the selected physiological measurement records successfully forwarded to the EMR system 102 are deleted for the physiological monitor device 200.

When a determination is made at operation 1518 that the physiological monitor device 200 is not configured for auto-delete of successfully sent physiological measurement records, at operation 1522 an acknowledgment symbol 1304 is displayed for each physiological measurement record successfully forwarded to the EMR system 102. The acknowledgment symbol 1304 is an envelope. However, other acknowledgment symbols may be used.

At operation 1524, one or more of the physiological measurement records displayed on the review screen 1300 may be manually deleted. To manually delete a physiological measurement record, a checkbox, for example checkbox 1306, is checked for each physiological measurement record to be deleted. The physiological measurement records selected by checking the checkbox are deleted when the delete button 1310 is pressed.

At operation 1526, all physiological measurement records are auto-deleted after 24 hours.

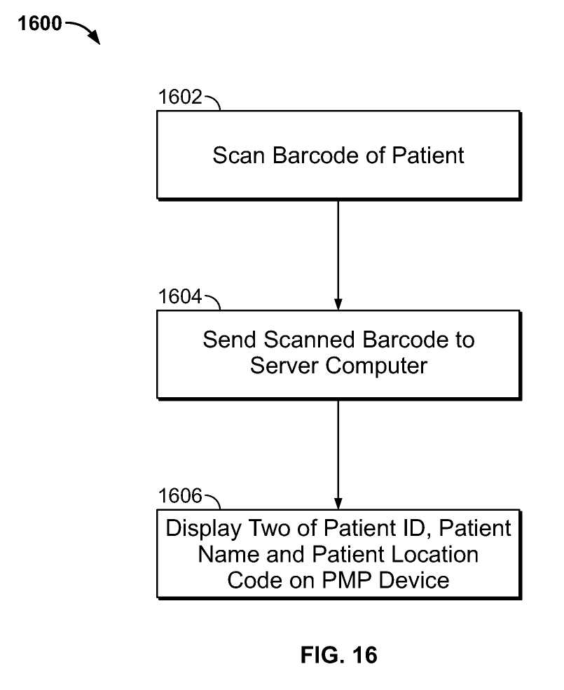

FIG. 16 is a flowchart illustrating an example method 1600 for displaying patient identification based on the scan of a barcode. At operation 1602, a barcode associated with a patient is scanned. The barcode is typically on an identification wristband worn by the patient. The barcode includes a patient ID that identifies the patient. The identification wristband is typically given to the patient when a patient checks in to a medical facility such as a hospital. A barcode scanner, typically attached to the physiological monitor device 200, scans the barcode.

At operation 1604, the scanned barcode is sent via the interface system 104. The interface system 104 maintains a list of patients populated from the EMR system 102. The interface system 104 uses the scanned barcode to lookup the patient in the database populated from EMR system 102 to determine the patient name and a location ID for the patient.

At operation 1606, two or more identifiers for the patient are displayed on the home screen, for example home screen 600 of the physiological monitor device 200. The two identifiers or more identifiers are selected from the patient name, the patient location and the patient ID. In the example home screen 600, the patient ID 604 corresponds to a primary label and the location ID 606 corresponds to a secondary label. In other examples, the primary label, as selected in example screen 500, may be the patient name or the location code. In other examples, the secondary label, as selected in screen 500, may be set to the patient name instead or the location ID. The display of the patient ID, the patient name and the location ID on home screen 600 as a result of a barcode scan of a patient identification wristband, provides a multiple means of identifying the patient for a clinician.

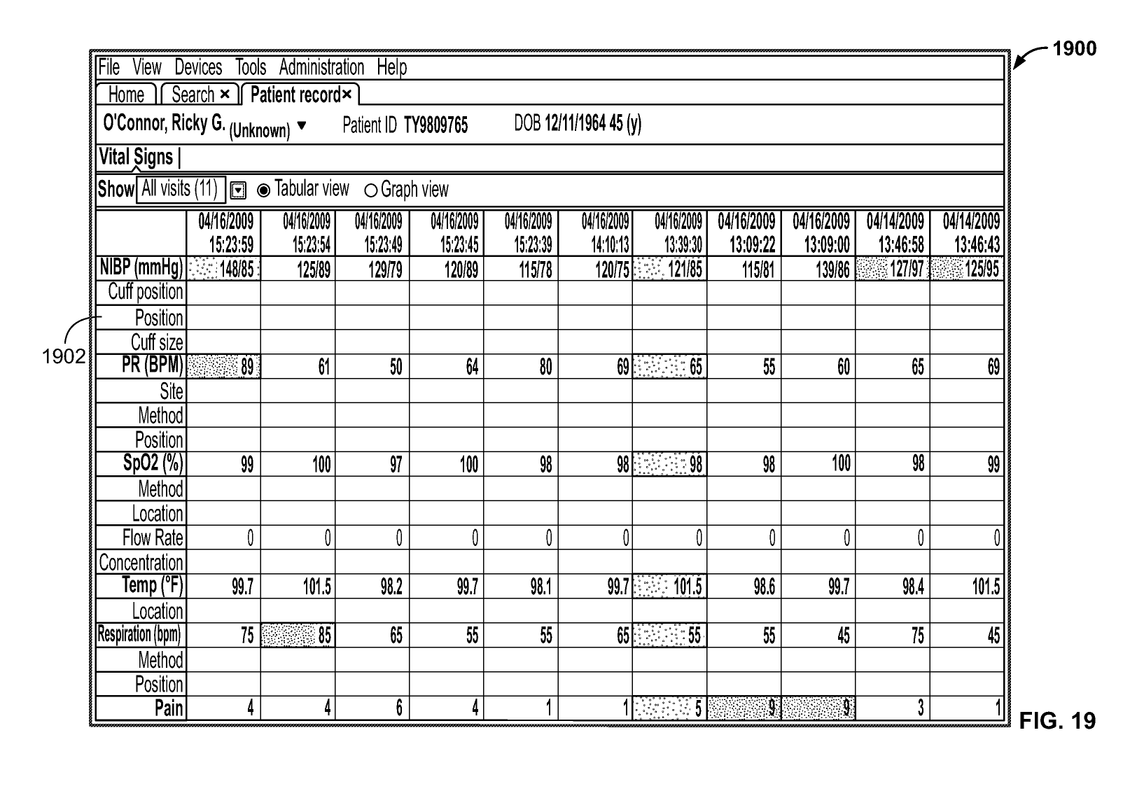

Referring now to FIG. 19, an example patient record 1900 is shown. The record 1900 displays one or more parameters measurement. The data for the physiological measurements is obtained by the physiological monitor device 200 sending a query to the interface system 104. The interface system 104, in turn, returns physiological measurement data for the identified patient from its database to the physiological monitor device 200 and displayed as physiological measurement data 1902 on the patient record 1900. In this manner, the clinician can use the physiological monitor device 200 to obtain historical physiological measurement data to allow for trending of patient physiological measurements at the point-of-care. In one example, data for one, two, five, ten, or another configurable number of readings is returned to the physiological monitor device 200 for display.

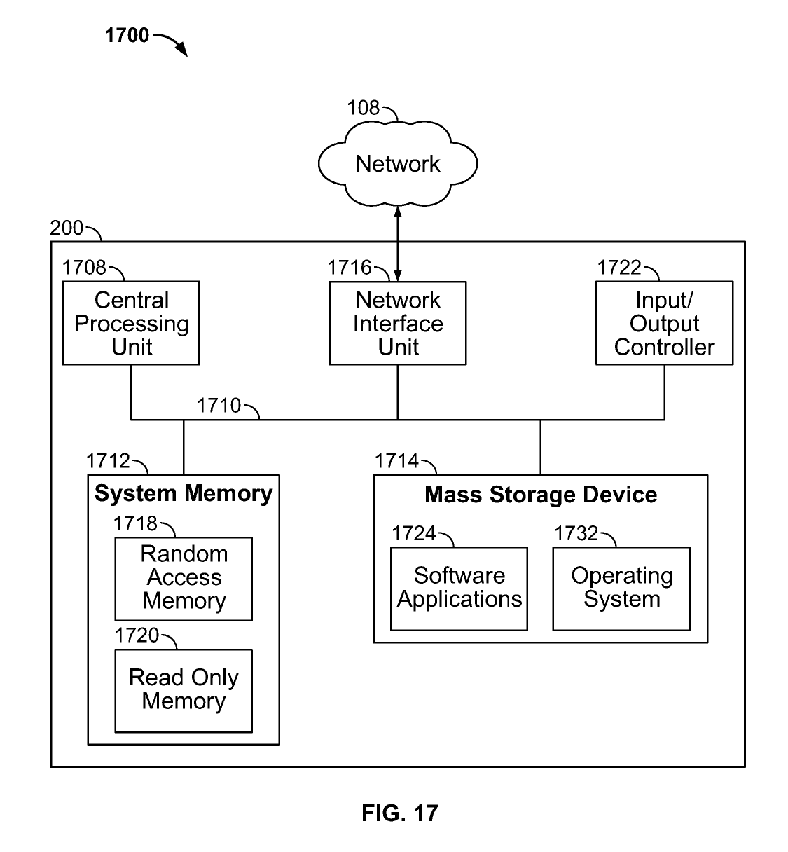

FIG. 17 illustrates example physical components of the physiological monitor device 200. As illustrated in the example of FIG. 17, the physiological monitor device 200 include at least one central processing unit (“CPU”) 1708, a system memory 1712, and a system bus 1710 that couples the system memory 1712 to the CPU 1708. The system memory 1712 includes a random access memory (“RAM”) 1718 and a read-only memory (“ROM”) 1720. A basic input/output system containing the basic routines that help to transfer information between elements within the physiological monitor device 200, such as during startup, is stored in the ROM 1720. The physiological monitor device 200 further includes a mass storage device 1714. The mass storage device 1714 is able to store software instructions and data.

The mass storage device 1714 is connected to the CPU 1708 through a mass storage controller (not shown) connected to the bus 1710. The mass storage device 1714 and its associated computer-readable data storage media provide non-volatile, non-transitory storage for the physiological monitor device 200. Although the description of computer-readable data storage media contained herein refers to a mass storage device, such as a hard disk or CD-ROM drive, it should be appreciated by those skilled in the art that computer-readable data storage media can be any available non-transitory, physical device or article of manufacture from which the physiological monitor device 200 can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the physiological monitor device 200.

According to various embodiments of the invention, the physiological monitor device 200 may operate in a networked environment using logical connections to remote network devices through the network 108, such as a local network, the Internet, or another type of network. The physiological monitor device 200 connects to the network 108 through a network interface unit 1716 connected to the bus 1710. It should be appreciated that the network interface unit 1716 may also be utilized to connect to other types of networks and remote computing systems. The physiological monitor device 200 also includes an input/output controller 1722 for receiving and processing input from a number of other devices, including a keyboard, a mouse, a touch user interface display screen, or another type of input device. Similarly, the input/output controller 1722 may provide output to a touch user interface display screen, a printer, or other type of output device.

As mentioned briefly above, the mass storage device 1714 and the RAM 1718 of the physiological monitor device 200 can store software instructions and data. The software instructions include an operating system 1732 suitable for controlling the operation of the physiological monitor device 200. The mass storage device 1714 and/or the RAM 1718 also store software instructions, that when executed by the CPU 1708, cause the physiological monitor device 200 to provide the functionality of the physiological monitor device 200 discussed in this document. For example, the mass storage device 1714 and/or the RAM 1718 can store software instructions that, when executed by the CPU 1708, cause the physiological monitor device to display the home screen 600 and other screens.

It should be appreciated that various embodiments can be implemented (1) as a sequence of computer implemented acts or program modules running on a computing system and/or (2) as interconnected machine logic circuits or circuit modules within the computing system. The implementation is a matter of choice dependent on the performance requirements of the computing system implementing the invention. Accordingly, logical operations including related algorithms can be referred to variously as operations, structural devices, acts or modules. It will be recognized by one skilled in the art that these operations, structural devices, acts and modules may be implemented in software, firmware, special purpose digital logic, and any combination thereof without deviating from the spirit and scope of the present invention as recited within the claims set forth herein.

Although the invention has been described in connection with various embodiments, those of ordinary skill in the art will understand that many modifications may be made thereto within the scope of the claims that follow. For example, it should be appreciated that the screens illustrated in this document are merely examples and that in other embodiments equivalent screens can have different contents and appearances. Accordingly, it is not intended that the scope of the invention in any way be limited by the above description, but instead be determined entirely by reference to the claims that follow.

What is claimed is:

1. A physiological monitor device comprising:
a central processing unit (CPU) that is configured to control operation of the device;
a display screen; and
a set of one or more computer readable data storage media storing software instructions that, when executed by the CPU, cause the device to:
store a device ID for the device, the device ID uniquely identifying the device, the device being configured with the device ID by a user;
store a location ID for the device, the location ID identifying a location in a medical facility, the device being configured with the location ID by a user;
send the device ID and the location ID to a server computer;
after the device ID and the location ID are sent to the server computer, receive a list of patients for the location specified by the location ID; and
display the list of patients on the device.

2. The device of claim 1, wherein at least one of the patients in the list of patients is displayed using an abbreviated name.

3. The device of claim 1, wherein the software instructions, when executed by the CPU, further cause the device to:
display a review screen on the device, the review screen displaying one or more physiological measurement records for patients at the location specified by the location ID.

4. The device of claim 3, wherein the software instructions, when executed by the CPU, further cause the device to:
process a selection of one or more patient physiological measurement records from the review screen, the selection being made by a user of the device;
send the one or more patient physiological measurement records to a server computer;
receive an acknowledgment from the server computer that the one or more patient physiological measurement records were received at the server computer; and
after receiving the acknowledgment from the server computer, display an acknowledgment symbol on the device for each physiological measurement record sent to the server computer.

5. The device of claim 3, wherein the software instructions, when executed by the CPU, further cause the device to:
process a selection of one or more patient physiological measurement records from the review screen, the selection being made by a user of the device;
send the one or more patient physiological measurement records to a server computer;
receive an acknowledgment from the server computer that the one or more patient physiological measurement records were received at the server computer; and
after receiving the acknowledgment from the server computer, automatically delete the one or more patient physiological measurement records.

6. The device of claim 1, wherein the software instructions, when executed by the CPU, further cause the device to:
process a selection of a patient displayed on the list of patients, the selection being made by a user of the device;
when the selection is processed, display on the device, a home screen that displays ongoing physiological measurement readings for the patient, the home screen on the device also displaying at least two of a patient ID, a patient name and the location ID.

7. The device of claim 1, wherein the software instructions, when executed by the CPU, further cause the device to:
process a selection of a patient displayed on the list of patients; the selection being made by a user of the device;
when the selection is processed, display a screen on the device that permits a user to enter information into the device pertaining to the manner in which physiological measurement readings are captured for the patient.

8. The device of claim 7, wherein the information includes at least two of a patient position during a non-invasive blood pressure reading, the size of blood pressure cuff used during the non-invasive blood pressure reading, the location of the blood pressure cuff used during the non-invasive blood pressure reading, a SPO2 flow rate and a SPO2 concentration.

9. The device of claim 1, wherein the software instructions, when executed by the CPU, further cause the device to:
blackout at least a portion of the display after a time interval, the time interval being entered into the device by a user.

10. A method for reviewing and processing physiological measurement records, the method comprising:
storing a device ID for a physiological monitor device, the device ID uniquely identifying the physiological monitor device, the physiological monitor device being configured with the device ID by a user;
storing a location ID for the physiological monitor device, the location ID specifying a floor or a room number in a medical facility, the physiological monitor device being configured with the location ID by a user;
sending the device ID and the location ID to a server computer;
after the device ID and the location ID are sent to the server computer, receiving a list of patients at the location specified by the location ID; and
displaying the list of patients on the physiological monitor device.

11. The method of claim 10, further comprising:
selecting a review tab on the physiological monitor device;
when the review tab is selected, displaying a list of physiological measurement records for the patients at the location specified by the location ID, each physiological measurement record in the list of physiological measurement records including a patient name.

12. The method of claim 11, wherein at least one of the patient names is abbreviated.

13. The method of claim 11, further comprising selecting one or more of the physiological measurement records and sending the physiological measurement records to a server computer.

14. The method of claim 13, wherein an acknowledgment is displayed on the physiological measurement monitor for each of the physiological measurement records sent to the server computer

15. The method of claim 13, wherein the selected physiological measurement records are automatically deleted from the list of physiological measurement records after the selected physiological measurement records are sent to the server computer.

16. The method of claim 11, wherein the physiological measurement records are deleted from the physiological monitor device after 24 hours.

17. The method of claim 11, further comprising:
selecting a patient from the list of patients;
after a patient is selected, filtering out all physiological measurement records not associated with the patient so that such physiological measurement records are not displayed.

18. The method of claim 10, further comprising:
selecting a patient from the list of patients;
after a patient is selected, displaying an integrated summary of information about the patient, the integrated summary including the patient position during a non-invasive blood pressure reading, a size of a blood pressure cuff used during the non-invasive blood pressure reading and a location of the cuff during the non-invasive blood pressure reading.

19. The method of claim 10, further comprising:
selecting a patient from the list of patients;
after a patient is selected, displaying a home screen on the physiological monitor device, the home screen displaying current physiological measurement readings for the patient, the home screen including at least two identifiers for the patient, the at least two identifiers including two of a patient ID, the name of the patient and a location ID for the patient.

20. The method of claim 10, further comprising:
sending a clinician identifier to the server computer;
after the clinician identifier is sent to the server computer, receiving a second list of patients at the location associated with the clinician identifier; and
displaying the second list of patients on the physiological monitor device.

21. The method of claim 10, further comprising:
sending a patient identifier to the server computer;
after the patient identifier is sent to the server computer, receiving one or more physiological measurements associated with the patient identifier; and
displaying the physiological measurements on the physiological monitor device.

22. A computer-readable storage medium comprising software instructions that, when executed, cause a physiological monitor device to:
store a device ID for the device, the device ID uniquely identifying the device, the device being configured with the device ID by a user;
store a location ID for the device, the location ID specifying a floor or a room number in a medical facility, the device being configured with the location ID by a user;
send the device ID and the location ID to a server computer;
after the device ID and the location ID are sent to the server computer, receiving a list of patients at the location specified by the location ID;
display the list of patients on the device;
select a patient from the list of patients;
after a patient is selected, display a home screen on the device, the home screen displaying current physiological measurement readings for the patient, the home screen including at least two identifiers for the patient, the at least two identifiers including two of a patient ID, the name of the patient and a location for the patient;
select a review tab on the device;
when the review tab is selected, display a list of physiological measurement records for the patients at the location specified by the location ID, each physiological measurement record in the list of physiological measurement records including a patient name, at least one patient name being abbreviated;
select one or more of the physiological measurement records and sending the physiological measurement records to a server computer; and
automatically delete the selected physiological measurement records from the list of physiological measurement records after the selected physiological measurement records are sent to the server computer.

23. A method for identifying a patient on a physiological monitor device, the method comprising:
scanning a barcode on a wristband attached to the patient, the barcode being scanned using a barcode scanning device;
sending the scanned barcode to a server computer;
receiving, at the physiological monitor device, a name of the patient associated with the scanned barcode; and
displaying, on the physiological monitor device, the patient name.
